(19) United States
(12) Patent Application Publication
(10) Pub. No.: US 2020/0360178 A1
(43) Pub. Date: Nov. 19, 2020

(54) METHOD FOR CREATING A STATISTICAL DATABASE FOR CONTROLLING A THERMAL THERAPY DEVICE AND A THERMAL THERAPY SYSTEM

(71) Applicant: Melinda SZEGEDI, Budapest (HU)

(72) Inventor: Melinda SZEGEDI, Budapest (HU)

(21) Appl. No.: 16/762,583

(22) PCT Filed: Sep. 20, 2018
(86) PCT No.: PCT/HU2018/050039

§ 371 Date: May 8, 2020

(30) Foreign Application Priority Data
Nov. 8, 2017 (HU) P1700449

(51) Int. Cl. A61F 7/02; G16H 20/30; G16H 40/67

(52) U.S. Cl. CPC A61F 7/02; A61F 2007/0075; G16H 40/67; G16H 20/30

(57) ABSTRACT

The object of the invention relates to a method for creating a statistical database for controlling a thermal therapy device (10), the essence of which is providing a remote server (200) suitable for sending and receiving data through an electronic communication channel, and for storing and processing the received data, and: measuring temperature ensured with the thermal therapy device (10), and a duration of treatment, and—receiving via an electronic data input unit (301) from the user: user data relating to physical parameters of the user, data relating to a position of the skin surface treated by the thermal therapy device (10), data suitable for characterising a pathological condition treated with the thermal therapy device (10)—linking the measured data and the received data to each other, and sending them to a remote server (200) through an electronic communication channel with a user interface (300), then—processing the sent data with the remote server (200), and building a statistical database using the results of the processing, and for subsequent treatments performed with the thermal therapy device (10) controlling the thermal therapy device (10) using the data of the statistical database.

The object of the invention relates to a method for creating a statistical database for controlling a thermal therapy device.

The object of the invention also relates to a thermal therapy system for the treatment of the pathological condition of the user.

One of the most common home-therapies frequently recommended by dentists, rheumatologists and traumatologists is the cooling or heating (thermal therapy) of the given body part of the patient. Depending on the nature of the pathological condition (inflammation, swelling, sprain, abscess, etc.), more intensive or moderate cooling or heating is recommended with the short-term application of a cold or hot water compress.

In traditional thermal therapy it is the most usual to use an ice pack taken out of the freezer or a salt pack heated up in the kitchen cooker, which may also cause freezing or burn injuries. A further disadvantage of the above methods is that the desired temperature is difficult to set, and that they are not suitable for maintaining a given temperature for an extended duration in a stable way.

The above problem is effectively overcome by devices containing a cooling-heating Peltier unit that operates on the basis of the Peltier principle. Patent document number US 2008/141681 discloses a device and method that regulates personal temperature. In the case of the presented solution the surface of the skin is cooled using a Peltier unit built in to an item of clothing or other accessory (such as a hat) worn by the user. The Peltier unit is arranged in a flexible and soft medium (e.g. polyester, polyurethane or other foam material) that ensures even distribution of the heat. The temperature of the surface of the skin is monitored by thermometer, thereby ensuring an even temperature. Patent document number U.S. Pat. No. 9,029,736 discloses a device worn by the user that regulates the temperature of the skin. This solution is capable of both cooling and heating, in accordance with the pre-set temperature. Temperature regulation is performed by a control module operated by the user. The presented device may be built into items of clothing or into other wearable accessories (e.g. bracelet). The cooling/heating unit in the case of this solution also operates on the basis of the thermoelectric principle.

Although the solutions presented above are able to ensure the desired temperature at a stable value for an extended period of time, they are primarily designed for improving the comfort of the user, and not for therapeutic purposes. The cooling and heating temperature, as well as the period of operation are adjusted by the user as he/she desires.

The great disadvantage of the currently known thermal therapy solutions is that they do not contain a precise guide in connection with the temperature to be applied during the therapy, the duration and extent of the therapy, and the pauses to be observed between therapies. Precisely because of this thermal therapy is recommended as merely a supplementary therapy for the treatment of pathological conditions.

It was recognised that at the present time there is no database in existence on the basis of which the parameters of the thermal therapy (temperature, duration of treatment, etc.) can be adjusted in accordance with the nature of the pathological condition.

It was also recognised that the efficacy of the thermal therapy treatments could be significantly increased and the wider use of thermal therapies could be facilitated with such a database.

The objective of the invention is the provision of a method for the creation of a database for controlling a thermal therapy device and a thermal therapy system that are free of the disadvantages of the solutions according to the state of the art. The object of the invention is especially to provide a method and system with the help of which a database may be created that links the characteristics of pathological conditions and the parameters of the required thermal therapy to the physical parameters of the user.

The invention is based on the recognition that by using the temperature provided by the thermal therapy device, the data obtained by measuring the duration of the treatment, and the data requested and received through the electronic data input unit from the users during the therapy a statistical database may be created with the help of which the result of the thermal therapy may be significantly improved.

The invention is also based on the recognition that with the help of a system containing a remote server having such a statistical database and a thermal therapy device having a wireless data transmission connection to it, the parameters required for operating the thermal therapy device can be set in a simple and effective way, and, additionally the reliability of the statistical database can be continuously developed.

In accordance with the above recognition the task was solved with the method according to claim 1 and with the thermal therapy system according to claim 9.

The preferred embodiments of the invention are determined in the subclaims.

Further details of the invention will be explained by way of exemplary embodiments with reference to figures, wherein:

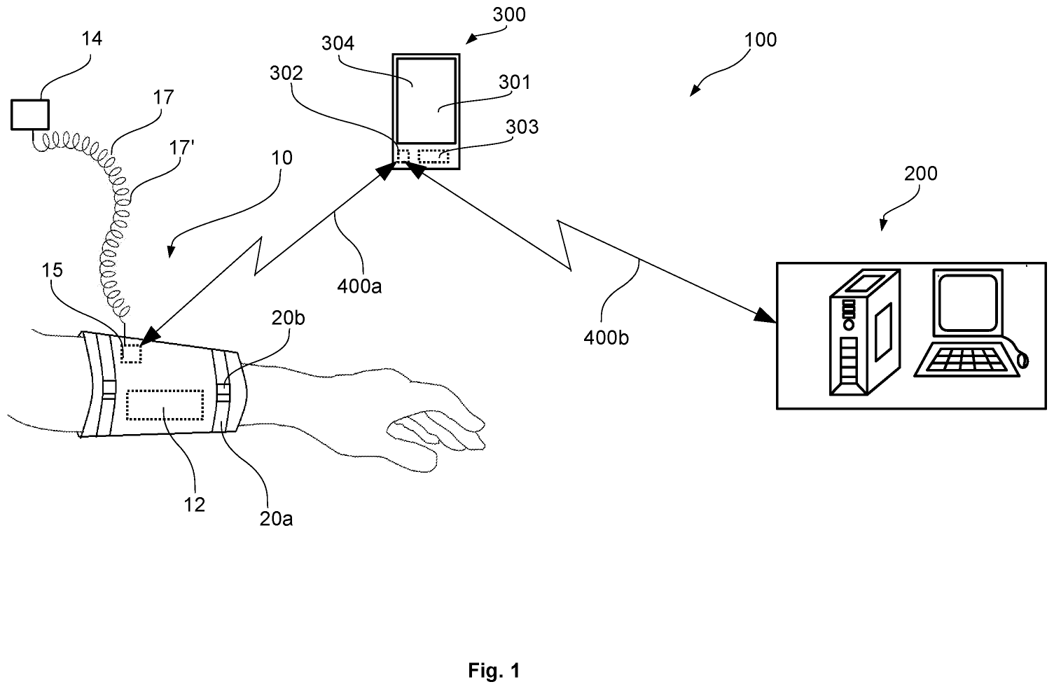

FIG. 1 shows a schematic view of a first embodiment of the thermal therapy system according to the invention,

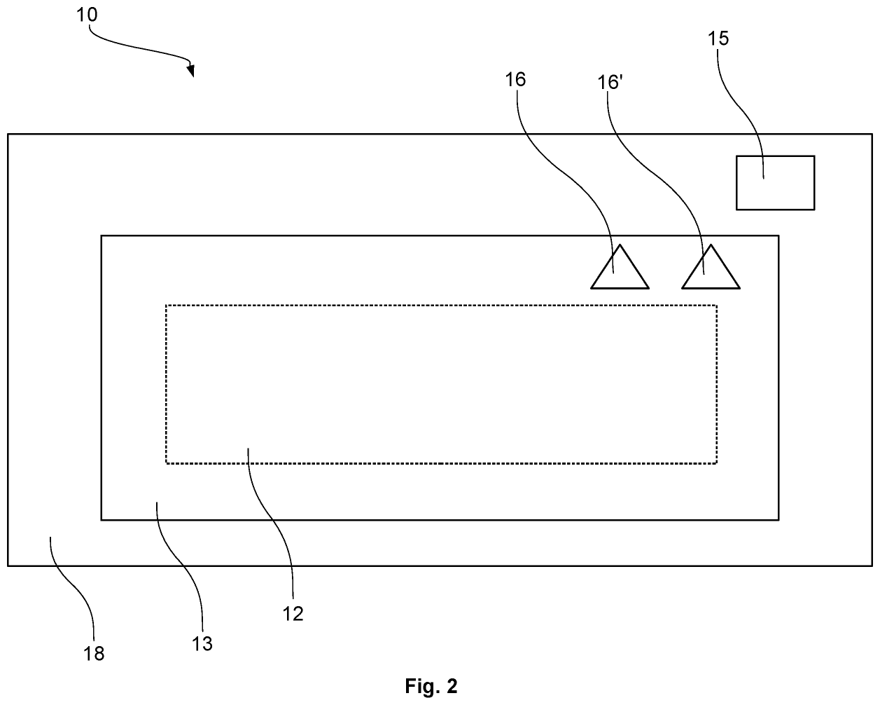

FIG. 2 depicts a schematic view of a preferred embodiment of the thermal therapy device according to the invention,

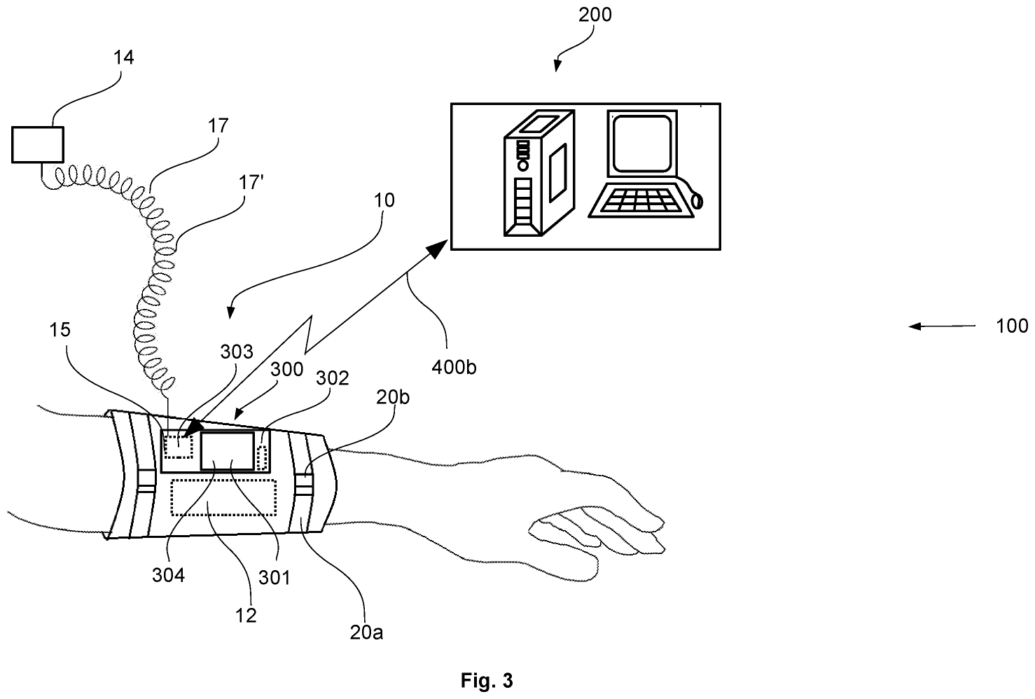

FIG. 3 shows a schematic view presenting a second embodiment of the thermal therapy system according to the invention.

The main elements of a preferred embodiment of the thermal therapy system 100 according to the invention are shown in FIG. 1.

The system 100 according to the invention contains a thermal therapy device 10 established in a way suitable for being in contact with the surface of the user's skin and for maintaining the temperature of the surface of the user's skin within a predetermined temperature range. The thermal therapy device 10 serves for performing thermal treatment of the pathological condition of the user. In the context of the present invention pathological condition means a physical change affecting the user's body that is usually treated by the heating, or, optionally, cooling of the surface of the skin of the user in medical practice. For the purpose of clarity it must also be noted that in the present specification user is understood to mean the user of the thermal therapy device 10, in other words the person treated with thermal therapy. In the case of a preferred embodiment the thermal therapy device 10 contains one or more cooling-heating Peltier units 12 operating on the basis of the Peltier principle, preferably flexible Peltier units 12′. The Peltier unit 12 heats up or cools down depending on the direction of the direct current conducted through it, as is known by a person skilled in the art. It should also be noted that, optionally, an embodiment is conceivable in the case of which the thermal therapy device 10 contains a heating element instead of the Peltier unit 12. Naturally, in this case, the thermal therapy device 10 is only suitable for heating.

The thermal therapy device 10 according to the invention is established in a way suitable for sending and receiving data in a wireless way, and in such a way so it can be wirelessly controlled. The thermal therapy device 10 is provided with control electronics 15 that are remotely operated with the help of electromagnetic waves and serve for regulating the direction and magnitude of the current passing through the Peltier unit 12. Such control electronics 15 may be established, for example, by using a microcontroller or system on chip (SoC) suitable for sending, receiving and processing data, as is known by a person skilled in the art.

In the case of the embodiment presented in FIG. 2, the thermal therapy device 10 contains a liquid pouch 13 in contact with the one or more Peltier units 12, which is positioned between the Peltier units 12 and the surface of the skin of the user, and prevents direct contact between the Peltier units 12 and the skin surface. The liquid pouch 13 is a vessel filled with a liquid, such as water, or, optionally a gel that is harmless to health and that has good heat-transmittance, which takes on the cooling or heating effect created by the Peltier unit 12 and distributes it homogenously, and also equalises the temperature fluctuations arising during switching the Peltier unit 12 on and off. If the wall of the vessel is made of a flexible, easily deformable material, such as plastic, the liquid pouch 13 may contain bracing elements, such as metal or plastic springs, that support the liquid pouch 13 during use (not shown).

The thermal therapy device 10 also preferably contains one or more thermometers 16 serving for determining the temperature of the skin of the user, and/or one or more impedance meters 16′ for determining the electrical conductivity of the skin of the user. The thermometer 16 may be established as a thermistor, for example, the electrical resistance of which varies as a function of temperature. The thermometer 16 and the impedance meter 16′ are preferably arranged on the liquid pouch 13.

The thermal therapy device 10 contains a device housing 18 serving for accommodating the Peltier units 12, the liquid pouch 13 and the control electronics 15, which device housing 18 is preferably established as a heat insulating bag made from a skin-friendly material such as solid sponge or terry fabric. The thermal therapy device 10 is preferably supplied with a strap 20a and releasable fixing element 20b, such a hook and loop closure or buckle, serving for fixing the thermal therapy device 10 to the user's body, with the help of which a suitably tight contact can be ensured between the user's skin surface and the thermal therapy device 10.

In the case of an exemplary embodiment the system 100 contains a battery 14, preferably a lithium battery, suitable for supplying the electricity required for the operation of the Peltier units 12. The battery 14 is preferably arranged remotely from the thermal therapy device 10, such as in a bag that may be fixed to the user's body, and is connected to the thermal therapy device 10 via an electric cable 17, preferably a spiral cable 17′. It should be noted that optionally embodiments are conceivable in the case of which the battery 14 is established as a part of the thermal therapy device 10, established integrally with it, or the power supply for the thermal therapy device 10 is provided via an electric cable, from the mains electricity supply.

The thermal therapy system 100 according to the invention also contains a remote server 200 suitable for wirelessly sending, receiving, processing and storing data. In the present specification the server 200 is understood to mean a computer or group of computers that have the customary hardware (central processing unit, data storage unit, communication unit), and the software components operating this hardware (operating system, firmware, etc.) known of by a person skilled in the art. A statistical database is stored on the data storage unit of the remote server 200, the function of which will be explained at a later point.

The thermal therapy device 10 contains a user interface 300 suitable for creating a wireless data connection with the remote server 200 and with the thermal therapy device 10. The user interface 300 is an information technology device that has an electronic data input unit 301 serving for inputting user data relating to the user's physical parameters, data relating to the position of the skin surface treated with the thermal therapy device 10, and data suitable for characterising the pathological condition treated with the thermal therapy device 10, a network unit 302 serving for receiving and sending the data sent by the thermal therapy device 10 and the remote server 200, a computing unit 303 serving for processing and storing the data, and an output unit 304 serving for displaying the data. The data input unit 301 may be, for example, a keyboard, a specially designed series of buttons, or preferably a touch screen, which is suitable for the user to input data. The output unit 304 is understood to mean an electronic device that is suitable for displaying data. The concept of display in the case of the present invention is interpreted broadly, in other words it is understood to mean the generation of audio signals in addition to visual signals. In the case of a particularly preferred embodiment, the output unit 304 is established as a touch screen that is also suitable for inputting data, therefore the output unit 304 also serves as the data input unit 301. The computing unit 303 serving for processing and storing the data includes the customary information technology components (such as a processor, memory, hard disc, SSD, SoC, etc.) known of to a person skilled in the art that are suitable for executing compute programs. The computing unit 303 has a data connection with the data input unit 301 and is capable of processing the inputted data and, preferably, capable of storing them. Furthermore, the computing unit 303 has a control connection with the output unit 304. The network unit 304 is understood to mean the sum total of the hardware and software (such as network card, network connector, Wi-Fi adapter, antenna, etc.), with the help of which the user interface 300 is able to create a first wireless communication channel 400a with the thermal therapy device 10, and a second wireless communication channel 400b with the server 200, and perform electronic data transmission through them. Such wireless communication channels 400a, 400b may be established between two points, such as with the help of a Bluetooth connection, infrared connection, NFC connection, etc., but may also be established within the framework of a communications network, which may be, for example, a wired and/or wireless local information technology network (LAN), or global information technology network, especially the Internet, a mobile telecommunications network according to the 3G or 4G standard, or a GSM network, etc. The network unit 302 has a data connection with the computing unit 303, which processes the data received by the network unit 302, and, optionally, stores this data. A control connection may be established through the wireless communication channel 400a between the user interface 300 and the thermal therapy device 10 using the network unit 302, more precisely through the computing unit 303 and the control electronics 15.

Therefore, in accordance with the above, the user interface 300 is established so as to be able to send the data inputted using the input unit 301, and received from the thermal therapy device 10 through the wireless communication channel 400a to the remote server 200, to download data from the database stored on the server 200 and to control the thermal therapy device 10 with the use of the downloaded data. In the case of a preferred embodiment the user interface 300 is a mobile device that makes it possible to control the thermal therapy device 10 on the basis of data received from the remote server 200, preferably a smartphone. It must be noted that, optionally, the user interface 300 may be, for example, a computer, tablet computer, PDA, etc.

The embodiment of the thermal therapy system 100 according to the invention shown in FIG. 3 only differs from that presented above in that the user interface 300 is established as a part of the thermal therapy device 10, constructed integrally with it. In the case of this embodiment the control electronics 15 of the thermal therapy device 10 and the computing unit 303 of the user interface 300 may even be established as a single, common unit.

The object of the invention also relates to the creation of a statistical database serving for controlling the thermal therapy device 10.

In the following the operation of the thermal therapy system 100 will presented simultaneously with the presentation of the method according to the invention.

During the method according to the invention a remote server 200 suitable for sending and receiving data through an electronic communications channel, such as the previously disclosed second wireless communication channel 400b, and for storing and processing the received data, as well as a thermal therapy device 10 are provided. The thermal therapy device 10 serves for treating the pathological condition of the user with thermal therapy, and is established in a way suitable for being in contact with the surface of the skin of the user and for maintaining the temperature of the skin surface of the user within a predetermined temperature range. The thermal therapy device 10 is fixed to the ill body part (or, optionally, to another body part that has a physiological relationship with the ill body part) using the strap 20a and the releasable fixing elements 20b on the device housing 18 in such a way that a suitably tight contact is established between the liquid pouch 13 and the skin surface.

In the case of a preferred embodiment, a thermal therapy device 10 is provided that is connected to a user interface 300 that is suitable for measuring the temperature of the surface of the skin of the user and the duration of the treatment, that serves for inputting and displaying data, and that has a wireless data connection with a remote server 200, and the electronic data input unit 301 is provided as a part of the user interface 300. The user sets the desired operation parameters (temperature, operation duration) of the thermal therapy device 10 using the data input unit 301 of the user interface 300. In the case of a preferred embodiment the user interface 300 is a mobile device, preferably a smartphone, on which a computer program (mobile app) performing the control of the thermal therapy device 10 runs. The touchscreen of the smartphone simultaneously functions as the data input unit 301 and as the output unit 304. In the case of this embodiment the task of the computing unit 303 is performed by the system on chip (SoC) and memory of the smartphone, and the wireless communication channel 400a is preferably provided with Bluetooth connection.

During the method according to the invention the temperature ensured by the thermal therapy device 10 and the duration of treatment are measured. The temperature ensured by the thermal therapy device 10 is preferably understood to mean the temperature of the skin surface of the user changed by the Peltier unit 12, which is preferably directly measured using the thermometer 16. The concept of measurement is also understood to mean indirect measurement, in other words, for example the case in which the temperature ensured by the thermal therapy device 10 is calculated in the knowledge of the magnitude of the current flowing through the Peltier unit 12 and the direction of the current. The duration of treatment is determined by the measurement of the duration of operation of the thermal therapy device 10.

The following step of the method includes
receiving user data relating to the physical parameters of the user,
receiving data relating to the position of the skin surface treated by the thermal therapy device 10,
receiving data suitable for characterising the pathological condition treated with the thermal therapy device 10,
   from the user via the electronic data input unit 301, such as the touch screen of a mobile device.
It should be noted that the expression “data” may also optionally mean a combination of different data types.

In the case of a preferred embodiment the user data relating to the physical parameters of the user are selected from among the group of data consisting of user identifier, body weight, height, age, sex, and skin type data. The user data are preferable provided during a registration process, to which the application running on the mobile device links a unique user identifier. Subsequently it is sufficient for the user to enter the allocated identifier using the data input unit 301. During registering the user may also define a password. Preferably real name, e-mail address are not recorded, so anonymity is ensured. With respect to that the thermal therapy device 10 may also be used by a family or healthcare institution, it is only necessary to identify the persons within these communities. Optionally an embodiment is conceivable in the case of which the application running on the mobile device operating as the user interface 300 may only be used after the user identifier and password have been entered, making it compulsory through this for the user to be identified.

The data relating to the position of the skin surface treated with the thermal therapy device 10 may be, for example, the name of the body area containing the treated skin surface, such as: upper arm, lower arm, torso, head, which may be optionally be further refined (such as middle part of upper arm, etc.).

The data suitable for characterising the pathological condition is preferably selected from the group consisting of data stating the type of pathological condition, its location, size, depth, as well as the state of well-being and the feeling of pain related to the user's pathological condition, but optionally other subjective data suitable for characterising the pathological condition, or objective data, such as the degree of advancement of the pathological condition, the extent of the inflammation, etc. may also enter into consideration. In the case of a particularly preferred embodiment, several options are displayed in accordance with the data relating to the position of the treated skin surface and/or data suitable for characterising the pathological condition with the use of the output unit 304 provided as a touch screen, and the user may select (e.g. by clicking) those options that, according to the user, best correspond to his/her actual condition. For example, a scale of from zero to ten is displayed on the touch screen functioning as both output unit 304 and data input unit 301 as the data suitable for characterising pathological condition, on which the user may indicate his/her feeling of pain relating to the pathological condition on the scale of from zero to ten, where zero indicates the lack of pain, and ten indicates the most intense pain. Similarly a list may be displayed relating to the type of pathological condition using the output unit 304, which, for example, lists the names of the possible illnesses (such as sprained ankle, back pain, rheumatoid left elbow, etc.) per category or in alphabetical order, in this way it is sufficient for the user to select the type of illness that corresponds to, or is most similar to his/her pathological condition. It should be noted that optionally an embodiment may be conceived in the case of which the data suitable for characterising the pathological condition (such as size, extent of the pathological condition) are entered by the member of the medical staff (e.g. doctor, nurse, etc.) treating the user instead of or in unison with the user with the data input unit 301.

The data relating to the temperature measured by the thermal therapy device 10 and the operation duration, as well as the data entered by the user are linked to each other and are sent to the remote server 200 via the electronic communication channel, such as the wireless communication channel 400b. The sending of the data preferably takes place with the user interface 300. In the context of the present invention linking the data to each other means an action or series of actions with which it may be ensured that the data sent via the communication channel correspond to each other on the server 200 side. The linking of the data may take place in several ways, for example in such a way that the data are linked into a single data package, and then this data package is sent. Another possible method of linking the data to each other is if the data are assigned an identifier, or if the data are sent at the same time. In the latter case the time of sending the data or of their arrival will be the common identifier of the data, on the basis of which the data will correspond to each other on the server 200 side.

In the case of a preferred embodiment the time data relating to the point in time of the treatment with the thermal therapy device 10 are determined, and then the time data along with the data relating to temperature and operation duration are sent to the remote server 200 via the electronic communication channel. The advantage of this is that not only can the duration of treatment be determined but also the time passing between subsequent treatments can be determined in the case of several, consecutive treatments.

The sent data are processed using the server 200, and a statistical database is built by using the result of the processing. In the context of the present invention statistical database is understood to mean the set of data measured by the thermal therapy device 10 and the data entered by the user along with their logical connection that is suitable for being searched in. The generation of the database may take place with known programs, such as the open source R-Statistics program, as is known of by a person skilled in the art. Preferably, during every use or following each use, the thermal therapy device 10 sends the data entered by the user and measured by the thermal therapy device 10 to the server 200, therefore by using the continuously arriving data the database may be continuously expanded. The system 100 preferably contains several thermal therapy devices 10, therefore the server 200 may receive data from several thermal therapy devices 10. By increasing the amount of the data received by the server 200, the complexity of the statistical database can be continuously increased; thereby the therapeutic efficacy of the thermal therapy device 10 can be determined with increasing precision.

In the case of a preferred embodiment the database contains the characteristics of the various pathological conditions, as the treatment parameters belonging to the given pathological condition that have proven to be the most effective (temperature, treatment duration, treatment frequency, etc.), in other words the data relating to the efficacy of the thermal therapy device 10. The user has access to the database stored on the server 200 via the user interface 300, therefore the user is able to download information corresponding to the current pathological condition. The user may set the optimal parameters of the thermal therapy device 10 him/herself on the basis of the downloaded information, thereby increasing the efficacy of the thermal therapy device 10.

During the method according to the invention the thermal therapy device 10 is controlled on the basis of the data of the statistical database for subsequent treatments performed with the thermal therapy device 10. In the case of a particularly preferred embodiment the process of downloading and the setting of the parameters of the thermal therapy device 10 takes place automatically as follows. By using the user interface 300 the user enters the characteristics of the pathological condition, in the previously presented way, for example. In other words this involves
receiving user data relating to the user's physical condition,
receiving data relating to the position of the skin surface treated with the thermal therapy device 10, and
   receiving data suitable for characterising the pathological condition treated with the thermal therapy device 10 from the user through the electronic data input unit 301. The measured data and the received data are then linked to each other, then subsequently the data are sent to the server 200 using the user interface 300 through the wireless communication channel 400b, then the data received are compared with the stored database. Treatment parameter data are then determined as a result of this comparison. The result of the comparison (such as the database entry that best corresponds to the data received) is sent by the server 200 to the user interface 300, which processes the data received, and automatically starts to control the thermal therapy device 10 on the basis of the received data. In other words the thermal therapy device 10 is controlled with the user interface 300 on the basis of the received treatment parameter data.

Using the thermal therapy system 100 according to the invention the user is able to treat his/her pathological condition much more effectively and simply as compared to the solutions available to date. A further advantage of the system 100 is that medical supervision is not required for its use; what is more, optionally the system 100 is able to completely replace medical decision-making. Using the method according to the invention the thermal therapy device may be controlled effectively, and through this the efficacy of the treatment may be increased.

Various modifications to the above disclosed embodiments will be apparent to a person skilled in the art without departing from the scope of protection determined by the attached claims.

1. Method for creating a statistical database for controlling a thermal therapy device (10), which thermal therapy device (10) serves for treating a pathological condition of a user with thermal therapy, and which thermal therapy device (10) is suitable for being in contact with a skin surface of the user and for maintaining a temperature of the user's skin surface within a predefined temperature range, characterised by, during each of several treatments performed with one or more thermal therapy devices (10):
measuring temperature ensured with the thermal therapy device (10), and a duration of treatment, and
receiving via an electronic data input unit (301) from the user:
user data relating to physical parameters of the user,
data relating to a position of the skin surface treated by the thermal therapy device (10),
data suitable for characterising a pathological condition treated with the thermal therapy device (10)
linking the measured data and the received data to each other, and sending them to a remote server (200) through an electronic communication channel with a user interface (300), then
processing the sent data with the remote server (200), and building a statistical database using the results of the processing, and for subsequent treatments performed with the thermal therapy device (10) controlling the thermal therapy device (10) using the data of the statistical database.

2. Method according to claim 1, characterised by, for subsequent treatments performed with the thermal therapy device (10) receiving from the user through the electronic data input unit (301):
user data relating to the user's physical parameters,
data relating to the position of the skin surface treated with the thermal therapy device (10), and
data suitable for characterising the pathological condition treated with the thermal therapy device (10)
transmitting the received data to the remote server (200) through the electronic communication channel with the user interface (300),
comparing by the server (200) the data received with the statistical database, and,
sending data forming a result of the comparison to the user interface (300) through the electronic communication channel, and processing the received data with the user interface (300), and performing automatic control of the thermal therapy device (10) on the basis of the received data.

3. Method according to claim 1 or 2, characterised by providing the electronic data input unit (301) as an integral part of the user interface (300).

4. Method according to any of claims 1 to 3, characterised by providing a thermal therapy device (10) that is adapted for wirelessly sending and receiving data and that may be wirelessly controlled, and providing the user interface (300) as a mobile device, preferably as a smartphone, and remotely controlling the thermal therapy device (10) with the user interface (300).

5. Method according to any of claims 1 to 4, characterised by providing the electronic communication channel within the framework of a global information technology network, preferably the Internet.

6. Method according to any of claims 1 to 5, characterised by selecting user data from a group consisting of user identifier, body weight, height, age, sex, and skin type data.

7. Method according to any of claims 1 to 6, characterised by selecting data suitable for characterising pathological condition from a group consisting of data on the position, size and depth of the pathological condition, as well as the data on the feeling of well-being and pain of the user related to the pathological condition.

8. Method according to any of claims 1 to 7, characterised by determining time data relating to the date of treatment performed with the thermal therapy device (10), and sending the time data to the remote server (200) via the electronic communication channel.

9. Thermal therapy system (100) especially for the implementation of the method according to claims 1 to 8, which thermal therapy system (100) comprising a thermal therapy device (10) for being in contact with the skin surface of a user, and for maintaining the skin surface of the user within a predefined temperature range, characterised by that the thermal therapy device (10) is adapted for wirelessly sending and receiving data and for being wirelessly controlled, and the thermal therapy system (100) further comprising:
a remote server (200) suitable for the wireless sending, receiving, processing and storage of data, on which remote server (200) a statistical database is stored, and
a user interface (300) suitable for establishing wireless data connection with the remote server (200) and the thermal therapy device (10),
said user interface (300) has:
an electronic data input unit (301) serving for inputting:
user data relating to the user's physical parameters,
data relating to the position of the skin surface treated with the thermal therapy device (10), and
data suitable for characterising the pathological condition treated with the thermal therapy device (10),
a network unit (302) serving for receiving and sending data sent by the thermal therapy device (10) and the remote server (200),
a computing unit (303) serving for processing and storing the data, and
an output unit (304) serving for displaying the data,
and said user interface (300) is configured for sending the inputted data and data received from the thermal therapy device (10) to the remote server (200), and for downloading data from a database stored on the remote server (200) and for controlling the thermal therapy device (10) using the downloaded data.

10. Thermal therapy system (100) according to claim 9, characterised by that the user interface (300) is a mobile device preferably a smartphone, that allows for controlling the thermal therapy device (10) on the basis of the data received from the remote server (200).

11. Thermal therapy system (100) according to claim 9 or 10, characterised by that it contains one or more thermometers (16) for determining the temperature of the skin surface of the user, and/or impedance meters (16′) for determining the electrical conductivity of the user's skin.

12. Thermal therapy system (100) according to any of claims 9 to 11, characterised by that the thermal therapy device (10) contains one or more cooling-heating Peltier units (12), preferably flexible Peltier units (12′).

13. Thermal therapy system (100) according to claim 12, characterised by that it contains a battery (14) ensuring the operation of the one or more Peltier units (12), preferable a lithium battery, and the battery (14) is arranged remotely from the thermal therapy device (10), which battery (14) is connected to the thermal therapy device (10) with an electric cable (17), preferably a spiral cable (17′).
